(19) United States
(12) Patent Application Publication
(10) Pub. No.: US 2016/0160224 A1
(43) Pub. Date: Jun. 9, 2016

(54) CRYPTOSPORIDIUM TRANSFECTION METHODS AND TRANSFECTED CRYPTOSPORIDIUM CELLS

(71) Applicant: UNIVERSITY OF GEORGIA RESEARCH FOUNDATION, INC., Athens, GA (US)

(72) Inventors: Boris Striepen, Athens, GA (US); Sumiti Vinayak, Athens, GA (US); Carrie F. Brooks, Nicholson, GA (US)

(73) Assignee: UNIVERSITY OF GEORGIA RESEARCH FOUNDATION, INC., Athens, GA (US)

(21) Appl. No.: 14/905,907

(22) PCT Filed: Aug. 1, 2014
(86) PCT No.: PCT/US2014/049386

§ 371 Date: Jan. 18, 2016

Related U.S. Application Data
(60) Provisional application No. 61/861,710, filed on Aug. 2, 2013.

(51) Int. Cl. C12N 15/79

(52) U.S. Cl. CPC C12N 15/79

(57) ABSTRACT

This disclosure describes, in one aspect, a method of transfecting a Cryptosporidium organism. Generally, the method includes introducing into a Cryptosporidium organism a heterologous polynucleotide comprising at least one coding region, and incubating the Cryptosporidium organism under conditions effective for the Cryptosporidium organism to express the coding region.

### CROSS-REFERENCE TO RELATED APPLICATION

This application claims priority to U.S. Provisional Patent Application Ser. No. 61/861,710, filed Aug. 2, 2013, which is incorporated herein by reference.

### GOVERNMENT FUNDING

This invention was made with government support under Grant No. UGARF CDC FID 795 (STRIEPEN) awarded by the UGA-CDC collaborative research program and RO1AI112427 awarded by the National Institutes of Health. The government has certain rights in the invention.

### SUMMARY

This disclosure describes, in one aspect, a method of transfecting a Cryptosporidium organism. Generally, the method includes introducing into a Cryptosporidium organism a heterologous polynucleotide comprising at least one coding region, and incubating the Cryptosporidium organism under conditions effective for the Cryptosporidium organism to express the coding region.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Vector map of a Cryptosporidium transfection plasmid.

FIG. 2. Luciferase activity in Cryptosporidium parvum transfected with a specific transfection plasmid or without DNA, a parallel experiment with the related parasite Toxoplasma gondii (a well-established genetic model) is shown for comparison.

FIG. 3. Expression of Nluc luciferase in C. parvum depends on parasite and not host cell transgenesis. Sporozoites were electroporated with an AMAXA NUCLEOFECTOR (Lonza Cologne GmbH, Cologne, Germany) device using indicated amounts of DNA (A) or number of parasites (B). Data shown reflect the mean of three experiments and the standard deviation. (C) Mock transfection omitting electroporation or parasites. Where the bars are too small to be visible, the mean values are shown, which are not significantly different from the no DNA control. (D) Electroporated parasites are grown in paramomycin. (E, F) C. parvum or T. gondii are electroporated with crypto or toxo_Nluc plasmid and used to infect HCT-8 cells. (G) HCT-8 cells are directly transfected with control, crypto and toxo-Nluc by lipofection (no parasites). Mean of three replicates is shown and error bars represent standard deviation.

FIG. 4. (A) Luciferase activity of C. parvum after transfection using AMAXA (Lonza Cologne GmbH, Cologne, Germany) or BTX ECM 630 (Harvard Apparatus, Inc., Holliston, Mass.) electroporation devices and protocols. (B) Transfection of C. parvum using the 4D NUCLEOFECTOR (Lonza Cologne GmbH, Cologne, Germany) was optimized using a combination of nucleofection buffers and electroporation settings. Sporozoites were prepared in the proprietary nucleofection buffer SF or SG and electroporated with 10 μg Crypto_Nluc plasmid. Eight electroporation programs were selected for optimization testing based on the manufacturer's suggestion (EH 100, EO 100, FA 100, DU 100, EN 100, ED 113, or DS 118). Transfection using cytomix buffer and BTX system was included for comparison. Electroporated parasites were used to infect cells and luciferase was measured as before.

FIG. 5. Luciferase activity using the upstream regulatory sequences of C. parvum enolase (Eno) or α-tubulin (Tub) genes.

FIG. 6. Luciferase activity in C. parvum recovered from the intestine of infected mice.

FIG. 7. Mouse surgery procedure to directly inject transfected sporozoites into the small intestine.

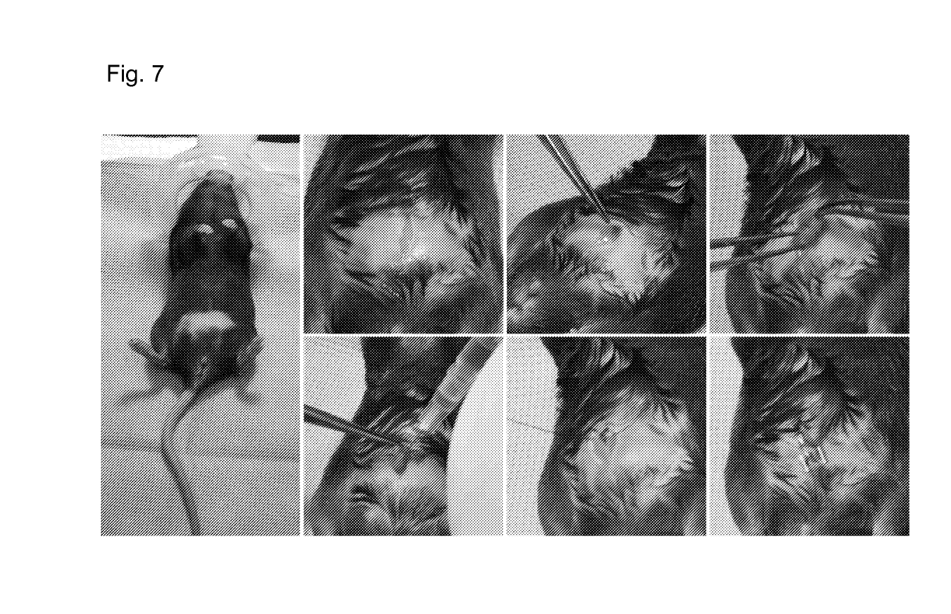

### DETAILED DESCRIPTION OF ILLUSTRATIVE EMBODIMENTS

Cryptosporidium is a genus of protozoans that can cause gastrointestinal illness with diarrhea in humans and in a variety of domestic animals. Indeed, Cryptosporidium infection is a common cause of diarrheal disease in infants. Unlike some other parasites, Cryptosporidium does not use an insect vector and is capable of completing its life cycle within a single host. Cryptosporidiosis is typically an acute, short-term infection, but can become severe in children and immunocompromised individuals. Cryptosporidium is also of particular veterinary concern for calves. The parasite is commonly transmitted in its spore phase through environmentally hardy cysts, called oocysts, that, once ingested, exist in the small intestine and result in an infection of intestinal epithelial tissue. Cryptosporidium oocysts can survive for lengthy periods outside a host and can resist many common disinfectants such as, for example, chlorine-based disinfectants

Currently, there are many challenges to studying Cryptosporidium including, for example, poor animal models, an inability to continuously grow the organism in culture, a lack of genetic tools, and the nature of Cryptosporidium as a poorly tractable pathogen. Moreover, there is no vaccine and very limited drug therapy available for treating infection by Cryptosporidium or Cryptosporidiosis.

In one aspect, this disclosure describes a method to transfect Cryptosporidium by electroporation of a DNA vector that includes heterologous DNA. We demonstrate use of the method to introduce a reporter DNA vector into a model Cryptosporidium species, C. parvum, and then to introduce the transgenic pathogens into the intestine of laboratory mice. We demonstrate transfection using a luciferase reporter assay.

In other aspects, this disclosure describes various applications of the Cryptosporidium transfection methods. Cryptosporidium transfectants can be used to establish reporter pathogens to, for example, measure infection, pathogenesis, efficacy of therapeutic treatments, and/or immunity to test drugs and vaccines and to more generally study the infection. Transfection can be used to modify the pathogen by, for example, gene knockout or other genetic changes to, for example, attenuate its pathogenicity and/or other biological parameters. This technology also can be used to, for example, engineer genetically-modified forms of the pathogen that may be suitable for use as an attenuated vaccine for people and animals such as, for example, livestock (e.g., calves). Transfection also can be used to introduce the ability to express additional antigens, which can lead to the development of a Cryptosporidium-based vaccine with the ability to also protect against additional diseases. Lastly, transfection allows the development of forward and reverse genetic tools to discover parasite genes involved in the development of drugs and vaccines and to establish their relative merit as a target.

In one aspect, this disclosure describes a method of transfecting Cryptosporidium with a heterologous polynucleotide. As used herein, a “heterologous” polynucleotide refers to a polynucleotide that does not naturally occur in the organism into which it is being introduced. The heterologous polynucleotide includes at least one coding region that can be expressed by the Cryptosporidium organism following transfection. As used herein, “coding region” refers to a nucleotide sequence that encodes a polypeptide so that, when placed under the control of appropriate regulatory sequences, the transfected Cryptosporidium organism expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Regulatory sequences include, for example, promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence. As used herein, “express” and variations thereof refer to the conversion of genetic information in a nucleotide sequence to a gene product. Expression of a nucleotide sequence (e.g., a coding region) may be measured and/or described with reference to (a) transcription of DNA to mRNA, (b) translation of mRNA to protein, (c) post-translational steps (e.g., modification of the primary amino acid sequence; addition of a carbohydrate, a lipid, a nucleotide, or other moiety to the protein; assembly of subunits; insertion of a membrane-associated protein into a biological membrane; and the like), or any combination of the foregoing.

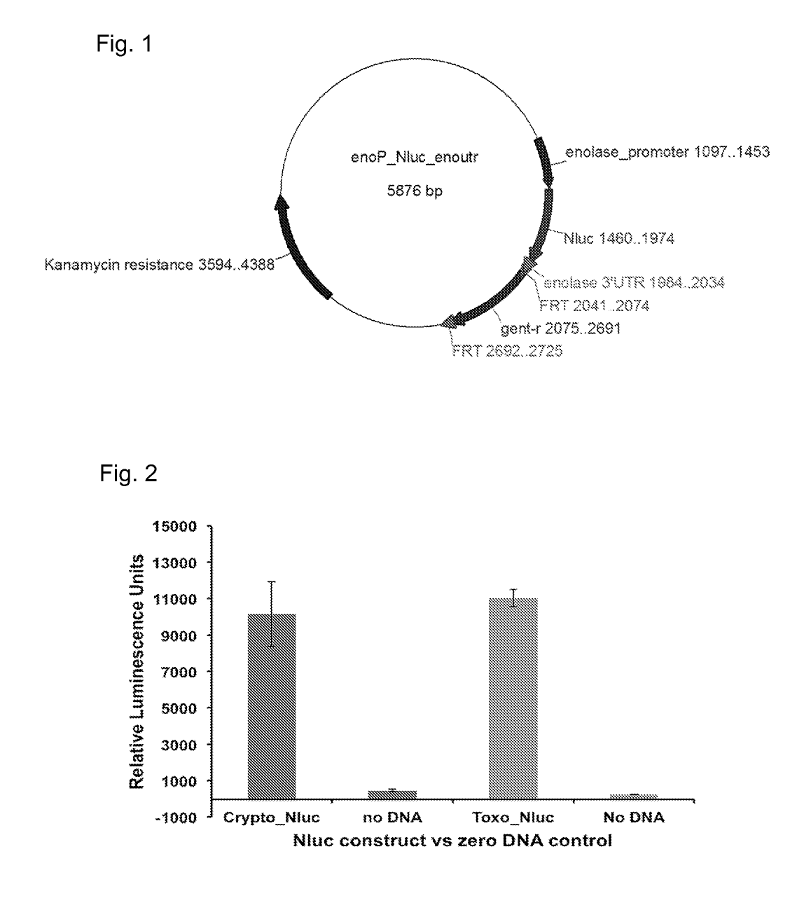

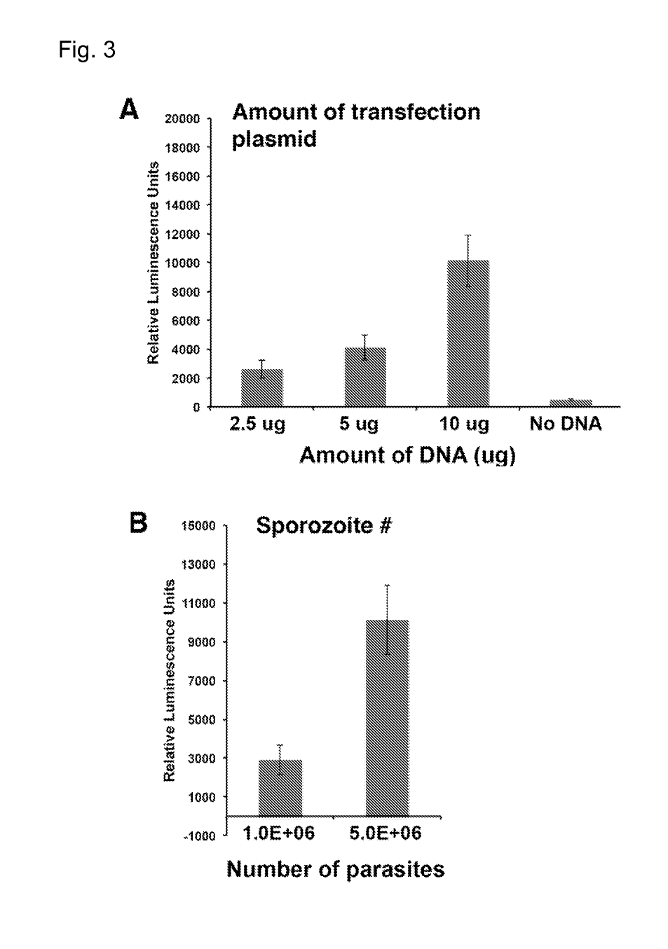

To introduce a transfection vector into Cryptosporidium, we started with C. parvum oocysts. We induced parasite excystation and purified the sporozoites by filtration. The transfection construct, shown in FIG. 1 includes a coding region for luciferase as a model heterologous polypeptide. The transfection construct was introduced into the C. parvum via electroporation. The electroporated sporozoites were then used to infect human ileocecal adenocarcinoma (HCT-8) cell cultures. Transfection was assessed by performing a luciferase assay after 48 hours of incubation to detect expression of the model heterologous polypeptide. We consistently detect luciferase activity in transfected parasites (FIG. 2, luciferase activity in the well-established genetic model parasite Toxoplasma gondii shown for comparison). We conducted a variety of control experiments (FIG. 2) and detected no activity in the absence of parasites, in the absence of DNA, or when we use a luciferase plasmid that lacks Cryptosporidium-specific sequence elements.

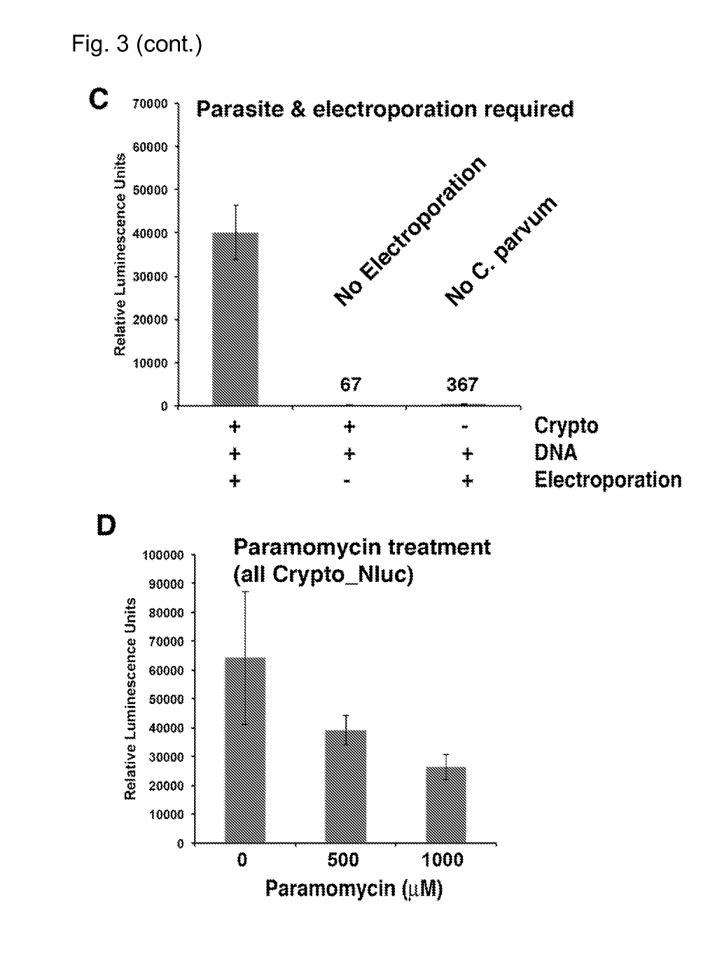

We next performed control experiments and varied the amount of transfection vector DNA or the number of sporozoites used in each electroporation. As shown in FIG. 3A and FIG. 3B, luciferase activity depends on both sporozoites and vector. To exclude the possibility of luciferase expression by host cells rather than the parasite, we performed a number of additional experiments. Mock transfections that were identical to the previous experiments but omitted parasite electroporation or the addition of parasites altogether showed no luciferase activity (FIG. 3C).

Next, we performed transfections and then cultured the transfected parasites in the presence of paramomycin, a drug that reduces growth of C. parvum but not the mammalian host cell. Paramomycin treatment results in a reduction of Nluc expression. (FIG. 3D). We also tested an Nluc plasmid that lacks the Cryptosporidium promoter but carries Toxoplasma flanking sequences instead. While this plasmid produces strong expression in T. gondii (FIG. 3F), it shows no activity in the C. parvum transfection assay (FIG. 3E). Conversely, electroporating the C. parvum vector into T gondii does not result in expression in that parasite (FIG. 3F), confirming species-specific parasite expression. Lastly, we tested whether parasite vectors can transduce host cells by deliberately delivering plasmid DNA into the HCT-8 host cell cytoplasm by lipofection. While we detect luciferase activity with the commercial Nluc plasmid that has suitable flanks (pNL1.1, Promega Corp., Madison, Wis.) and from which we originally amplified the Nluc gene, we do not detect activity when using our C. parvum or T gondii Nluc vectors (FIG. 3G). Collectively these measurements confirm that Apicomplexa and mammals have divergent primary sequence elements that drive transcription and subsequent RNA processing. Cryptosporidium transfection therefore depends on the introduction of a parasite species specific vector into viable parasites. Moreover, passive DNA delivery into the host is not relevant for activity and host cells are not transfected even when DNA is deliberately introduced into the host.

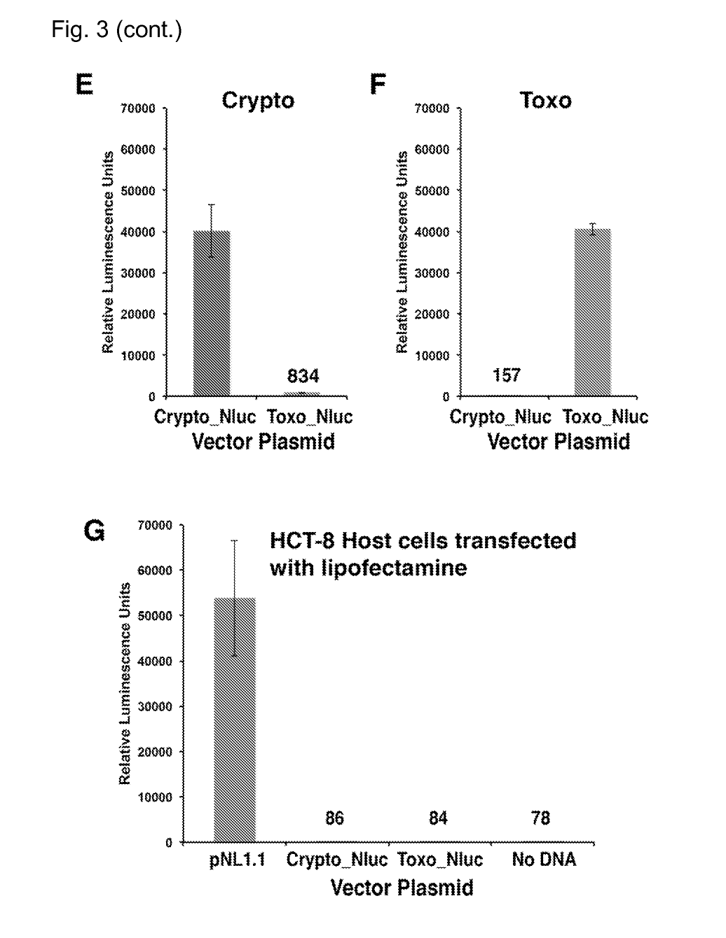

The heterologous DNA may be introduced into the Cryptosporidium using any suitable method such as, for example, electroporation, lipofection, bombardment, etc. We tested two different electroporation protocols using a BTX ECM 630 electroporator (Harvard Apparatus, Inc., Holliston, Mass.) and an AMAXA NUCLEOFECTOR device (Lonza Cologne GmbH, Cologne, Germany). For the BTX ECM 630 electroporator, the sporozoites were suspended in complete cytomix buffer, mixed with DNA, and electroporated with a single 1500V pulse, resistance of 25Ω, and a capacitance of 25 μF. For the AMAXA device, sporozoites were suspended in Human T-cell buffer and electroporation was conducted using the U33 program. The BTX electroporation protocol results in higher luciferase expression. (FIG. 4A). FIG. 4B shows transfection of C. parvum using a 4D-NUCLEOFECTOR device (Lonza Cologne GmbH, Cologne, Germany). Transfection with buffers SF and SG and electroporation programs EH 100 and ER 100 produced the highest luciferase readings.

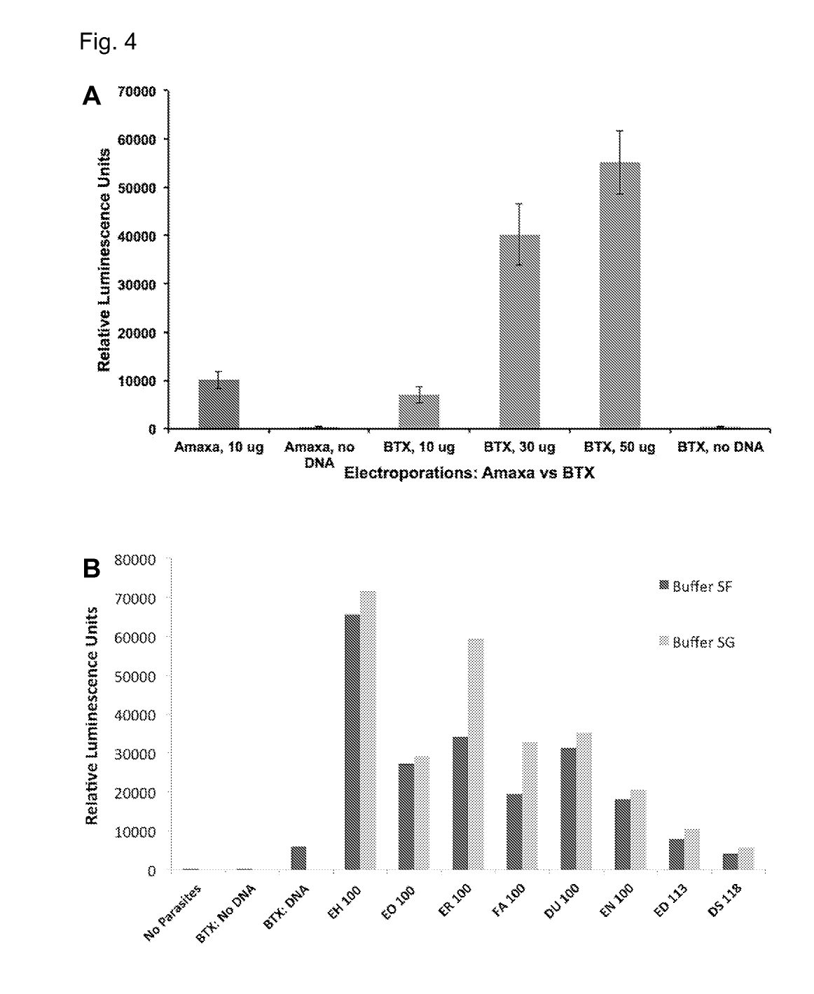

We also tested the efficiency of different Cryptosporidium regulatory and flanking sequences and found consistent differences in their ability to drive the transgenic reporter. FIG. 5 shows a comparison of the upstream regulatory regions of the a-tubulin and enolase genes. FIG. 5 shows that the enolase promoter is a stronger promoter than the tubulin promoter, driving higher luciferase expression in vitro.

Our in vitro methods can be adapted for in vivo transfections. As C. parvum cannot be grown continuously culture in vitro, this can involve infecting susceptible mice (e.g., interferon-γ knockout mice) with transfected sporozoites. Sporozoites typically infect poorly. In natural infection they are protected by the oocyst wall, which we have to remove for electroporation, from the stomach environment. Stable transgenesis can involve using, for example, a paramomycin resistance marker and drug selection, a cassette targeting the endogenous thymidine kinase locus, selection with a thymidine activated prodrug (e.g. triflurothymidin), and/or fluorescent protein expression and fluorescence activated cell sorting. Frequency of stable transformation may be enhanced by CRIPR/CAS9-mediated double stranded breaks in the thymidine kinase gene or other genomic regions. A suitable plasmid may, for example, place S. pyogenes CAS9 under the control of the C. parvum enolase promoter and a suitable guide RNA under the control of the C. parvum U6 promoter (genome contig CM000433 position 553110..553472).

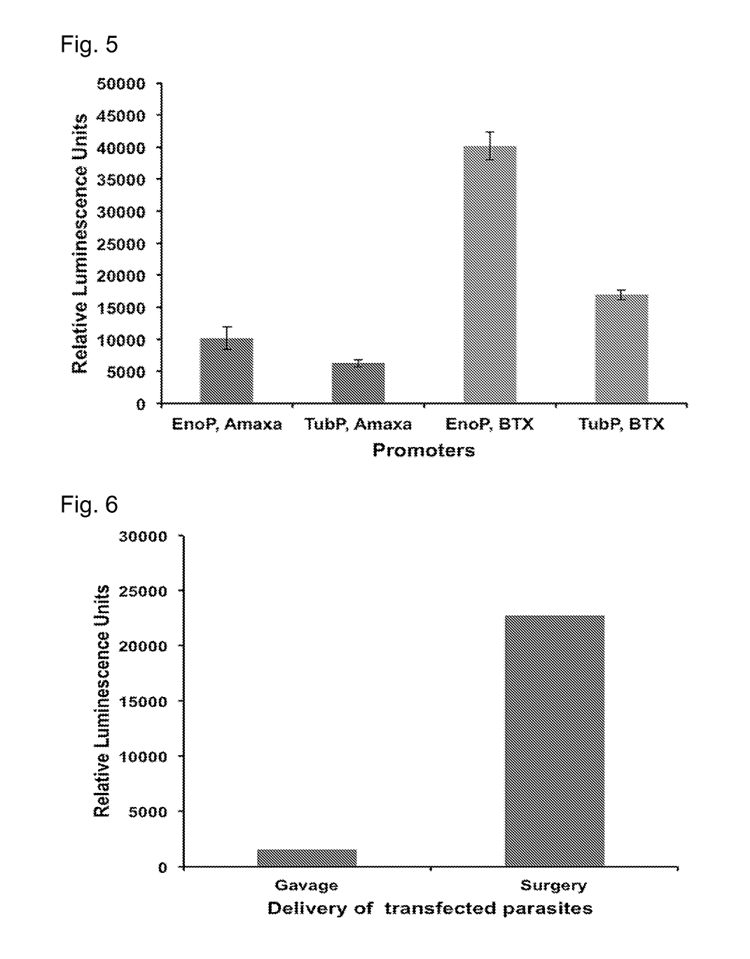

Transfected Cryptosporidium may be introduced to a subject using any suitable method such as, for example, oral administration, gavage, surgical placement, etc. We developed two procedures to introduce transfected Cryptosporidium sporozoites into mice with the long-term goal of stable transgenesis: gavage versus surgery. We compared both procedures by infecting mice with C. parvum sporozoites transfected with the luciferase plasmid. After 24 hours, mice were euthanized and the intestines were removed and flushed with saline. The small intestine was opened, the epithelium was scraped, and the scrapings were assayed for luciferase activity. FIG. 6 shows a representative result. We found both procedures to be effective, with surgery delivering higher luciferase activities. Alternatively, one could administer the transfected Cryptosporidium in an orally-ingestible form designed to deliver the transfected Cryptosporidium to the intestine of the subject. In some cases, this can involve encapsulating the transfected Cryptosporidium in material that will allow the transfected Cryptosporidium to pass through the stomach (and/or, for some subjects, the rumen), but will degrade on the subject's intestine sufficiently to release the transfected Cryptosporidium in the intestine.

Transfected Cryptosporidium organisms can be tools that have numerous applications. For example, Cryptosporidium organisms can be engineered to express a detectable signal and can thereafter function as a reporter parasite. Reporter parasites can facilitate study of infection and pathogen growth. Suitable detectable signals can include, for example, a visible signal such as, for example, a luciferase polypeptide, a β-galactosidase polypeptide, or fluorescent polypeptide or a colorimetric polypeptide that emits a signal detectable with or without the aid of detection instrumentation.

Other applications include testing of vaccines and/or therapeutic drugs. This can involve the use of transfection to test whether a particular Cryptosporidium coding region is required for pathogenesis, growth, spread, and/or infectivity of the pathogen. Exemplary Cryptosporidium coding regions include those that encode, for example, thymidine kinase, inosine monophosphate dehydrogenase, dehyrdofolate reductase, thymidylate synthase, polyketide synthase, tryptophan synthase B, and fatty acid synthase I.

Still other applications involve use as a vaccine component. In some cases, the Cryptosporidium organism can be modified so that its virulence is attenuated. Suitable Cryptosporidium organisms in this context can include, for example, those that can cause disease in, for example, humans. Thus, suitable Cryptosporidium organisms can include C. parvum, C. hominis, C. canis, C. felis, C. meleagridis, C. muris, C. tyzeri, C. andersoni, and C. bayleii. An attenuated Cryptosporidium organism can serve as a vaccine and/or as a therapeutic treatment. Moreover, an attenuated Cryptosporidium organism can be further transfected to include one or more heterologous antigens that may be expressed by the transfected Cryptosporidium organism. A heterologous antigen may serve as an adjuvant to, for example, increase a subject's immune response to the attenuated Cryptosporidium vaccine. In other cases, the heterologous antigen may provide protection against infection by a second pathogen (e.g., a bacterium, virus, or parasite), thereby producing a single attenuated vaccine that can provide protection against infection by multiple pathogens. This may be of particular value for pathogens that, like Cryptosporidium, cause intestinal infection. Exemplary pathogens thus include, for example, rotavirus, norovirus, enterotoxic E. coli, Shigella, Entamoeba, Campylobacter, Adenovirus, Salmonella, Vibrio cholerae, and Aeromonas.

In still other applications, transfected Cryptosporidium organism can permit genetic crossing experiments between parasites as a tool for discovering candidate genes for modification.

As used herein, the term “and/or” means one or all of the listed elements or a combination of any two or more of the listed elements; the terms “comprises” and variations thereof do not have a limiting meaning where these terms appear in the description and claims;

unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Construction of Transfection Vector

The pH₃BG vector was modified to clone in the promoter, luciferase gene, and 3′UTR elements. The C. parvum promoters for the enolase (cgd5_1960) and α-tubulin (cgd_2860) genes were PCR amplified from genomic DNA and cloned into the BamHI and NheI sites of the vector. The luciferase gene was amplified and cloned into the NheI and PacI sites, and the 3′ UTRs (enolase-51 by or α-tubulin-97 bp) were cloned into the PacI and AgeI sites of the vector. The 685 bp FRT-Gentamicin resistance-FRT cassette was PCR amplified from pH₃BG and cloned immediately downstream of the 3′ UTR in the AgeI and NotI sites. The vector also has a kanamycin resistance marker in the backbone.

### Parasite Excystation and Transfection

C. parvum oocysts were purchased from Sterling Parasitology laboratory (Tucson, Ariz.) or Waterborne Inc. (New Orleans, La.). Synchronous excystation was carried out using the method of Gut and Nelson (J Eukaryot Microbiol, 46, 56S-57S. 1999. PMID: 10519247) with some modifications in the protocol. Oocysts were surface-sterilized by adding 1 ml of 10 mM HCL and incubated on ice for 10 minutes. The oocysts were pelleted at 14000 rpm for three minutes at 4° C., supernatant was discarded and the pellet was washed with ice-cold phosphate-buffered saline (PBS), pH 7.2. This washing step with PBS was repeated three times to remove the HCl. The pellet was then suspended in 200 μl of 0.2 mM sodium deoxy taurocholate and incubated at 15° C. for 10 minutes in a water bath. After the incubation, the oocysts were incubated at 37° C. for one hour. Excystation of the sporozoites was checked by observing the parasites under the microscope, and if the excystation was incomplete, an additional incubation of oocysts for 20 minutes at 37° C. was done.

After excystation, the parasites were filtered through a 3 μM polycarbonate membrane filter to remove un-excysted oocysts, and the number of sporozoites obtained were counted. The sporozoites were then pelleted at 14000 rpm for three minutes at 4° C., and washed with 1 ml ice-cold PBS. For electroporation using a BTX ECM 630 electroporator (Harvard Apparatus, Inc., Holliston, Mass.), the washed sporozoites were suspended in complete cytomix buffer (120 mM KCl, 0.15 mM CaCl₂, 10 mM K₂HPO₄/KH₂PO4, pH 7.6, 25 mM HEPES, pH 7.6, 2 mM EGTA, 5 mM MgCl₂, pH 7.6 supplemented with 2 mM ATP and 5 mM glutathione), mixed with plasmid DNA and electroporated with a single 1500V pulse, resistance of 25Ω, and a capacitance of 25 μF. For electroporation using an AMAXA NUCLEOFECTOR device (Lonza Cologne GmbH, Cologne, Germany), the sporozoites were suspended in AMAXA Human T-cell buffer, mixed with DNA and electroporated using the U33 program. For electroporation using an AMAXA NUCLEOFECTOR 4D device (Lonza Cologne GmbH, Cologne, Germany), the sporozoites were suspended in AMAXA SG or SF buffer, mixed with DNA and electroporated using the EH100 program.

### Infection of HCT-8 Cells by Sporozoites (In Vitro)

A 60%-70% confluent monolayer of the human ileocecal adenocarcinoma epithelial cell line (HCT-8) was used to support C. parvum infection in vitro. HCT-8 cells were maintained in RPMI-1640 supplemented with 10% FBS, 1 mM sodium pyruvate, 50 U/ml penicillin, 50 μg/ml streptomycin, and amphotericin B, in T-25 flasks or 24-well plates. The HCT-8 media was removed prior to infection by C. parvum sporozoites, and replaced with infection medium (DMEM supplemented with 2% FBS, 50 U/ml penicillin, 50 μg/ml streptomycin, amphotericin B and 0.2 mM L-glutamine). The electroporated sporozoites were added to the HCT-8 host cells and infection was allowed to proceed at 37° C. for 48 hours. The media was removed after 24 hours of incubation, and replaced with fresh RPMI infection media.

### Infection of Mice by Sporozoites (In Vivo)

The C. parvum sporozoites were excysted and electroporated as described above, and used to infect C57BL/6 [KO] IFN-γ mice by oral gavage or surgery.

### Gavage

Thirty minutes prior to oral administration of 10 to 10⁷ purified C. parvum sporozoites, C57BL/6 [KO] IFN-gamma mice were given 200 μl of a solution of 1% sodium bicarbonate in sterile water by gavage using a 24G-1″ straight 1.25 mm ball stainless feeding needle. Gavage of sporozoites followed at 200 μl or less volume in the same manner as sodium bicarbonate administration.

### Surgery

Abdominal area of mice was shaved with clippers. Animals were placed in isofluorane (3-5%) anesthesia induction chamber and then moved to a nosecone (1-3% isofluorane as needed) on the sterile surgical field. Respiration and response to stimulation (toe pinch) was monitored during procedure and vaporizer adjusted as needed. Mucous membranes and foot pads remained a normal color indicating that the animal's perfusion was adequate. Three betadine scrubs followed by a 70% ethanol wipe was applied to shaved skin prior to all surgeries. Microbial contamination was minimized in all surgeries via performance of each procedure under aseptic techniques in defined surgical area/field. The surgery area was disinfected with 70% alcohol before use. A sterile drape was applied over a warming pad placed on the surgery surface. Mice were placed abdomen facing up on surgical drape. Ophthalmic ointment (PURALUBE, Dechra Veterinary Products, Shrewsbury, UK) was applied to prevent drying of eyes. Skin was vertically incised approximately 1.5 cm in length. The incision was made midline in the abdominal region below the sternum with microsurgical scissors. A midline to off midline, vertical incision approximately 1.25 cm in length of the peritoneum was carried out with microsurgical scissors.

Exposed jejunum/ileum was injected with 200 μl of transfected C. parvum sporozoites (<10⁷) with a sterile food coloring dye. The peritoneum was closed with polydioxanone (PDS) in a 4/0 size. Closure of the skin was done with 9 mm wound clips. Administration of 0.01-0.02 ml/gram body weight of either warm lactated Ringer's solution was given subcutaneously post surgery. Meloxicam analgesic was also administered to the mice post surgery. At completion of procedure, the eye ointment was wiped off and the vaporizer was turned off and the mice were allowed to breathe the oxygen supply gas until they begin to awaken. Mice were placed in a recovery area with thermal support until ambulatory and exhibiting normal respiration.

### Luciferase Assay

For in vitro experiments, cells were scraped from T-25 flasks or 24-well plates and collected after 48 hours of incubation. For in vivo experiments, mice were euthanized after 24 hours, and the small intestines were removed. The intestines were flushed with PBS, and the mucosal scrapings were collected. The cell scrapings were then lysed and suspended completely by pipetting and luciferase substrate was added. After five minutes of incubation at room temperature, the cell lysate was added to white 96 well plates and luminescence was measured on a luminometer (BioTek Instruments, Inc., Winooski, Vt.). The read out was quantified in terms of relative luminescence units (RLU).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
introducing into a Cryptosporidium organism a heterologous polynucleotide comprising at least one coding region; and
incubating the Cryptosporidium organism under conditions effective for the Cryptosporidium organism to express the coding region.

2. The method of claim 1 wherein the coding region encodes a detectable marker.

3. The method of claim 2 wherein the detectable marker comprises a luminescent peptide, a colorimetric polypeptide or a fluorescent polypeptide.

4. The method of claim 1 wherein the coding region encodes a heterologous antigen.

5. The method of claim 1 wherein the coding region encodes a polypeptide that inhibits growth or infectivity of the Cryptosporidium organism.
